(19) United States
(12) Patent Application Publication
(10) Pub. No.: US 2013/0164335 A1
(43) Pub. Date: Jun. 27, 2013

(54) METHODS AND COMPOSITIONS FOR CELLULAR DRUG RELEASE

(71) Applicants: Cheng CHEN, Taipei City (TW); Feng-Huei Lin, Miaoli (TW)

(72) Inventors: Cheng CHEN, Taipei City (TW); Feng-Huei Lin, Miaoli (TW)

(73) Assignee: NATIONAL HEALTH RESEARCH INSTITUTES, Miaoli County (TW)

(21) Appl. No.: 13/338,073

(22) Filed: Dec. 27, 2011

(51) Int. Cl. A61K 9/14; C12N 5/071; C12N 5/0787; C12N 5/0786; A61K 47/02; A61P 25/24; A61K 47/40; A61K 47/44; A61K 47/24; C12N 5/0784; B82Y 5/00

(52) U.S. Cl. 424/400; 514/784; 435/375; 514/769; 514/777; 977/700; 977/906

(57) ABSTRACT

Methods and compositions for producing a cellular drug release are disclosed. The method comprises: a) providing a composition comprising a therapeutically effective amount of a pharmacological agent adsorbed onto mesoporous hydroxyapatite (HAP) with hydrophobic surfaces; b) exposing the composition to a cell; c) causing entry of the mesoporous HAP into the cell and degradation of the HAP in the lysosomes of the cell and desorption of the agent from the mesoporous HAP; d) causing release of the desorbed agent from the lysosomes into the cytoplasm of the cell; and e) causing release of the desorbed agent to outside the cell. The composition comprises a) mesoporous HAP with hydrophobic surfaces; and b) a therapeutically effective amount of a pharmacological agent, adsorbed onto the hydrophobic surfaces of the mesoporous. HAP. The composition is characterized in that it constantly releases the agent in vivo for a period of at least 4 weeks.

### FIELD OF THE INVENTION

The present invention relates generally to controlled drug delivery, and more specifically to controlled drug release through cellular activities.

### BACKGROUND OF THE INVENTION

Currently most so-called sustained release drug formulations complete drug release within 2 to 3 days after injection and fail to achieve a long term sustained release effect, or have drug release in two stages with an intermittent pause of 2 weeks. For example, using PCL, PLA, PLGA to form a sphere, drug molecules not entrapped within the sphere are released at initial burst, which is followed by a pause of release for about 2 weeks. A second stage of drug release occurs as the sphere is hydrolyzed.

Therefore, a heretofore unaddressed need exists in the art to address the aforementioned deficiencies and inadequacies related to drug delivery formulations, especially in connection with long term sustained drug delivery.

### SUMMARY OF THE INVENTION

In one aspect, the invention relates to a composition comprising a therapeutically effective amount of a pharmacological agent adsorbed onto mesoporous hydroxyapatite (HAP) with hydrophobic surfaces.

In another aspect, the invention relates to a composition comprising: (a) mesoporous hydroxyapatite (HAP); (b) acrylic acid, grafted onto the surfaces of the mesoporous HAP and forming an acrylic acid-grafted mesoporous HAP; (c) linoleic acid, modifying the surfaces of the acrylic acid-grafted mesoporous HAP and forming hydrophobic hydrocarbon tails on the surfaces thereof; and (d) a therapeutically effective amount of a pharmacological agent adsorbed onto the hydrophobic hydrocarbon tails.

Further in another aspect, the invention relates to a method of producing a cellular drug release, comprising: (a) providing a composition comprising a therapeutically effective amount of a pharmacological agent adsorbed onto mesoporous hydroxyapatite (HAP) with hydrophobic surfaces; (b) exposing the composition to a cell; (c) causing entry of the mesoporous HAP into the cell and degradation of the mesoporous HAP in the lysosomes of the cell and desorption of the pharmacological agent from the mesoporous HAP; (d) causing release of the desorbed pharmacological agent from the lysosomes into the cytoplasm of the cell; and (e) causing release of the desorbed pharmacological agent to outside the cell.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIG. 1 shows X-ray diffraction (XRD) pattern of mesoporous HAP nanoparticles.

FIG. 2 shows a scanning electron microscope (SEM) image of mesoporous HAP (100 000×).

FIG. 3 is a graph showing differential Thermal Analysis-Thermogravimetric Analysis (TGA-DTA) results of (1) mesoporous HAP, (2) HAP-AA-LA, and (3) HAP-AA-LA-OLZ.

FIG. 4 is a graph showing result of LDH assay for 3T3 cell line.

FIG. 5 is a graph showing result of WST-1 assay for 3T3 cell line.

FIG. 6 is a graph showing in vitro drug release profile in cumulative mass.

FIG. 7 is a graph showing in vitro drug release profile in cumulative percentage.

FIG. 8 is a graph showing in vivo drug release profile.

FIG. 9 is a schematic drawing illustrating one embodiment of the invention.

FIG. 10 is a schematic drawing illustrating another embodiment of the invention.

### DETAILED DESCRIPTION OF THE INVENTION

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, when a number or a range is recited, ordinary skill in the art understand it intends to encompass an appropriate, reasonable range for the particular field related to the invention.

By having a size of ranging from 500 to 3700 nm, it meant that all integer unit amounts within the range are specifically disclosed as part of the invention. Thus, 500, 501, 502 . . . 1000, 1001, 1002 . . . 3697, 3698, 3699 and 3700 nm unit amounts are included as embodiments of this invention.

As used herein, the term “mesoporous hydroxyapatite (HAP)” shall generally mean hydroxyapatite (Ca₁₀(PO₄)₆(OH)₂) particles with a mesoporous structure.

Amphiphile is a term describing a chemical compound possessing both hydrophilic (water-loving, polar) and lipophilic (fat-loving, hydrophobic) properties. Such a compound is called amphiphilic or amphipathic.

A fatty acid is a carboxylic acid with a long unbranched aliphatic tail (chain), which is either saturated or unsaturated. Fatty acids have a hydrophobic tail and hydrophilic head. A hydrophobic tail usually consists of long fatty acid hydrocarbon chains. Fatty acid chains differ by length, often categorized as short, medium, or long. Short-chain fatty acids (SCFA) are fatty acids with aliphatic tails of fewer than six carbons (i.e. butyric acid). Medium-chain fatty acid (MCFA) are fatty acids with aliphatic tails of 6-12 carbons, which can form medium-chain triglycerides. Long-chain fatty acid (LCFA) are fatty acids with aliphatic tails longer than 12 carbons. Very long chain fatty acid (VLCFA) are fatty acids with aliphatic tails longer than 22 carbons.

The terms “hydrophobic tail,” “hydrophobic hydrocarbon tail,” “hydrophobic tail region” and “lipophilic tail” are interchangeable, e.g., a hydrophobic (lipophilic) fatty acid tail (chain).

Commonly pharmaceutically acceptable ionic or nonionic surfactants include sodium lauryl sulfate (SLS), polyoxyethylenesorbitan monolaurate (Tween), cetyltrimethylammoniumbromide (CTAB), polyoxyl castor oil (Cremophor), hexadecyltrimethylammonium bromide (HTAB), polyethylene glycol tert-octylphenyl ether (Triton), nonylphenol ethoxylate (Tergitol), cyclodextrins, and lecithin.

Adsorption is the adhesion of atoms, ions, biomolecules or molecules of gas, liquid, or dissolved solids to a surface. The term “adsorb” means to undergo or cause to undergo a process in which a substance accumulates on the surface of a solid forming a thin film.

A physiological salt solution is a solution of a salt or salts that is essentially isotonic with tissue fluids or blood; especially: an approximately 0.9 percent solution of sodium chloride—called also normal saline solution, normal salt solution, physiological saline solution, physiological salt solution.

The term “treating”, “treat” or “treatment” as used herein includes preventative (e.g., prophylactic) and palliative treatment.

By “pharmaceutically acceptable” is meant the vehicle, carrier, diluent, excipients, and/or salt must be compatible with the other ingredients of the formulation, and not deleterious to the recipient thereof.

The quantity and nature of the pharmaceutically appropriate vehicle, carrier, diluent, excipients, and/or salt can be easily determined by a person skilled in the art. They are chosen according to the desired pharmaceutical form and method of administration.

The term “HAP-AA-LA-OLZ” stands for a composite (or a nanocomposite) of mesoporous HAP-acrylic acid-linoleic acid-olanzapine.

Composite materials, often shortened to composites or called composition materials, are engineered or naturally occurring materials made from two or more constituent materials with significantly different physical or chemical properties.

In one aspect, the invention relates to a composition comprising a therapeutically effective amount of a pharmacological agent adsorbed onto mesoporous hydroxyapatite (HAP) with hydrophobic surfaces.

In another aspect, the invention relates to a composition comprising: (a) mesoporous hydroxyapatite (HAP); (b) acrylic acid, grafted onto the surfaces of the mesoporous HAP and forming an acrylic acid-grafted mesoporous HAP; (c) linoleic acid, modifying the surfaces of the acrylic acid-grafted mesoporous HAP and forming hydrophobic hydrocarbon tails on the surfaces thereof; and (d) a therapeutically effective amount of a pharmacological agent adsorbed onto the hydrophobic hydrocarbon tails.

Further in another aspect, the invention relates to a method of producing a cellular drug release, comprising: (a) providing a composition comprising a therapeutically effective amount of a pharmacological agent adsorbed onto mesoporous hydroxyapatite (HAP) with hydrophobic surfaces; (b) exposing the composition to a cell; (c) causing entry of the mesoporous HAP into the cell and degradation of the mesoporous HAP in the lysosomes of the cell and desorption of the pharmacological agent from the mesoporous HAP; (d) causing release of the desorbed pharmacological agent from the lysosomes into the cytoplasm of the cell; and (e) causing release of the desorbed pharmacological agent to outside the cell.

In one embodiment of the invention, the aforementioned composition exhibits the following characteristics: i) release of less than 10% of the pharmacological agent from the HAP in a fluid having a pH value of about 7.4; and ii) release of the pharmacological agent in a fluid having a pH value of about ≦5.

In one embodiment of the invention, the cell is present in an animal.

In another embodiment of the invention, the desorbed pharmacological agent in step (e) is released into the blood stream of the animal.

In another embodiment of the invention, the desorbed pharmacological agent is released into the blood stream of the animal continuously for a period of 4 weeks or longer than 4 weeks without an intermittent cessation.

In another embodiment of the invention, the desorbed pharmacological agent is released into the blood stream of the animal continuously for a period of 5 weeks or longer than 5 weeks.

In another embodiment of the invention, the cell in the animal is exposed to the composition via an intramuscular route.

In another embodiment of the invention, the cell comprises neutrophils, monocytes, macrophages, dendritic cells, and mast cells.

In another embodiment of the invention, the aforementioned method further comprises causing an increase in Ca²⁺ and PO₄³⁻ ions within the lysosomes of the cell.

In another embodiment of the invention, the mesoporous HAP comprises HAP particles with each particle having a size of ranging from 500 to 3700 nm.

In another embodiment of the invention, the mesoporous HAP with hydrophobic surfaces comprises: a) mesoporous hydroxyapatite (HAP); b) acrylic acid, grafted onto the surface of the mesoporous HAP and forming an acrylic acid-grafted mesoporous HAP; and c) a pharmaceutically acceptable amphiphilic compound, modifying the surface of the acrylic acid-grafted mesoporous HAP and forming hydrophobic hydrocarbon tails on the surfaces thereof.

In another embodiment of the invention, the amphiphilic compound comprises a fatty acid from between 10 to 40 carbon atoms.

In another embodiment of the invention, the fatty acid is selected from the group consisting of capric acid, lauric acid, myristic acid, palmitic acid, palmitoleic acid, stearic acid, isostearic acid, elaidic acid, oleic acid, linoleic acid, polyunsaturated elaidolinoleic acid, polyunsaturated linolenic acid, elaidolinolenic acid, polyunsaturated ricinoleic acid, arachidic acid, behenic acid, erucic acid, lignoceric acid, ceric acid, montanic acid, melissic acid, and geddic acid.

In another embodiment of the invention, the amphiphilic compound comprises linoleic acid.

In one aspect, the invention relates to a drug delivery composition comprising: a) mesoporous hydroxyapatite (HAP); b) acrylic acid, grafted onto the surfaces of the mesoporous HAP and forming an acrylic acid-grafted mesoporous HAP; c) a pharmaceutically acceptable amphiphilic compound, modifying the surfaces of the acrylic acid-grafted mesoporous HAP and forming hydrophobic tails on the surface thereof; and d) a therapeutically effective amount of a pharmacological agent, adsorbed onto the surfaces of the amphiphilic compound-modified, acrylic acid-grafted mesoporous HAP.

The mesoporous HAP according to the invention comprises a wormlike structure.

In another aspect, the invention relates to a drug delivery composition comprising: a) mesoporous hydroxyapatite (HAP); b) acrylic acid, grafted onto the surfaces of the mesoporous HAP and forming an acrylic acid-grafted mesoporous HAP; and c) a pharmaceutically acceptable amphiphilic compound, modifying the surface of the acrylic acid-grafted mesoporous HAP and forming hydrophobic tails on the surface thereof.

Further in another aspect, the invention relates to a drug delivery composition comprising: a) mesoporous hydroxyapatite (HAP) having hydrophobic surfaces; and b) a therapeutically effective amount of a pharmacological agent, adsorbed onto the hydrophobic surface of the mesoporous HAP.

In one embodiment of the invention, the mesoporous hydroxyapatite (HAP) with the hydrophobic surfaces comprises: a) mesoporous hydroxyapatite (HAP); b) acrylic acid, grafted onto the surfaces of the mesoporous HAP and forming an acrylic acid-grafted mesoporous HAP; and c) a pharmaceutically acceptable amphiphilic compound, modifying the surfaces of the acrylic acid-grafted mesoporous HAP and forming hydrophobic hydrocarbon tails on the surface thereof.

In another embodiment of the invention, the composition further comprises a physiological saline, wherein the pharmacological agent exhibits a sustained release profile for a period of at least 5 weeks.

In another embodiment of the invention, the pharmacological agent exhibits a sustained release profile for a period of at least 6 weeks.

In another embodiment of the invention, the pharmacological agent exhibits a sustained release profile in vivo for a period of at least 5 weeks.

In another embodiment of the invention, the pharmacological agent exhibits a sustained release profile for a period of at least 7 weeks.

In another embodiment of the invention, the pharmacological agent exhibits an initial burst release of less than 10% of the agent adsorbed.

In another embodiment of the invention, the hydrophobic surfaces of the mesoporous HAP comprise fatty acid tails.

In another embodiment of the invention, the fatty acid comprises from between 16 to 26 carbon atoms.

In another embodiment of the invention, the fatty acid comprises a long chain fatty acid.

In another embodiment of the invention, the fatty acid comprises a very long chain fatty acid.

In another embodiment of the invention, the pharmacological agent comprises an antidepressant.

In another embodiment of the invention, the antidepressant comprises olanzapine.

Olanzapine, abbreviated in short as OLZ, is often used for the treatment of depression and schizophrenia. It possesses the most promising attributes due to its hydrophobic nature. Currently, commercially available OLZ is administered orally or intramuscularly once per day.

In another embodiment of the invention, the mesoporous hydroxyapatite (HAP) contains no water-soluble polyvalent metal compound and/or calcium as a constituent of hydroxyapatite is not substituted by other metal. According to the invention, the mesoporous HAP of the composition does not contain silica or polymer.

In another embodiment of the invention, the amphiphilic compound comprises a pharmaceutically acceptable surfactant.

In another embodiment of the invention, the surfactant comprises a polyoxyethylene glycolated natural or hydrogenated vegetable oil, or hydrogenated castor oil.

In another embodiment of the invention, the pharmaceutically acceptable surfactant is at least one selected from the group consisting of sodium lauryl sulfate (SLS), polyoxyethylenesorbitan monolaurate (Tween), cetyltrimethylammoniumbromide (CTAB), polyoxyl castor oil (Cremophor), hexadecyltrimethylammonium bromide (HTAB), polyethylene glycol tert-octylphenyl ether (Triton), nonylphenol ethoxylate (Tergitol), cyclodextrins, and lecithin.

In another embodiment of the invention, the pharmacological agent is hydrophobic.

In another embodiment of the invention, the composition further comprises a pharmaceutically acceptable vehicle, carrier, diluents and/or excipients.

Yet in another aspect, the invention relates to a formulation comprising a drug delivery composition as aforementioned, wherein the pharmacological agent exhibits a sustained release profile for a period of no shorter than 2 weeks.

In one embodiment of the invention, the pharmacological agent of the formulation exhibits a sustained release profile in vivo for a period of at least 4 weeks without an intermittent cessation.

The aforementioned surface-modified mesoporous HAP loaded with a drug (such as antidepressant) is prepared as an injectable form and administrated via intra-muscular injection. The HAP particles are taken up by defense cells (macrophage, dendritic cells, mast cells, phagocytes) in a living body and enters the lysosomes of the defense cells. The pH value of lysosomes is around 2-5, which can quickly dissolve mesoporous HAP particles and release the loaded drug to cytoplasm. The drug is then pumped out of the defense cells and delivered to the surroundings of the cells, and then diffuses to the local blood circulation system for therapeutics thereafter. The cellular activity lasts for about 4 weeks or longer until all the mesoporous HAP particles are taken up at the injection site. The drug release is thus controlled by the cellular activity of defense cells, and thereby achieves the goal of constantly daily drug release.

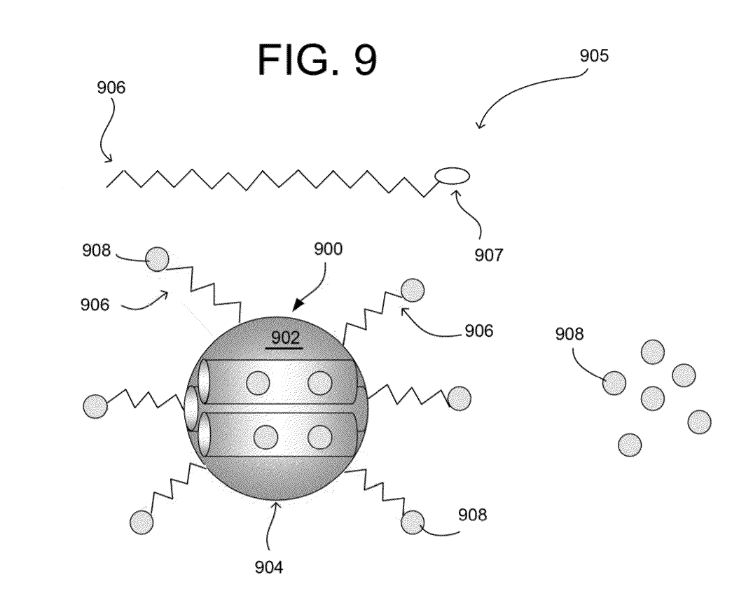

FIG. 9 is a schematic drawing illustrating one embodiment of the invention. A drug delivery system (composition) 900 having hydrophobic tails 906 comprises: (a) mesoporous HAP 902; (b) acrylic acid, grafted onto the surface of the mesoporous HAP 902 and forming an acrylic acid-grafted mesoporous HAP 904, (c) an amphiphilic compound 905, modifying the surface of the acrylic acid-grafted mesoporous HAP 904 and forming hydrophobic tails 906 on the surface thereof; and (d) a therapeutic agent 908. The amphiphilic compound 905 has a hydrophobic tail 906 and a hydrophilic head 907.

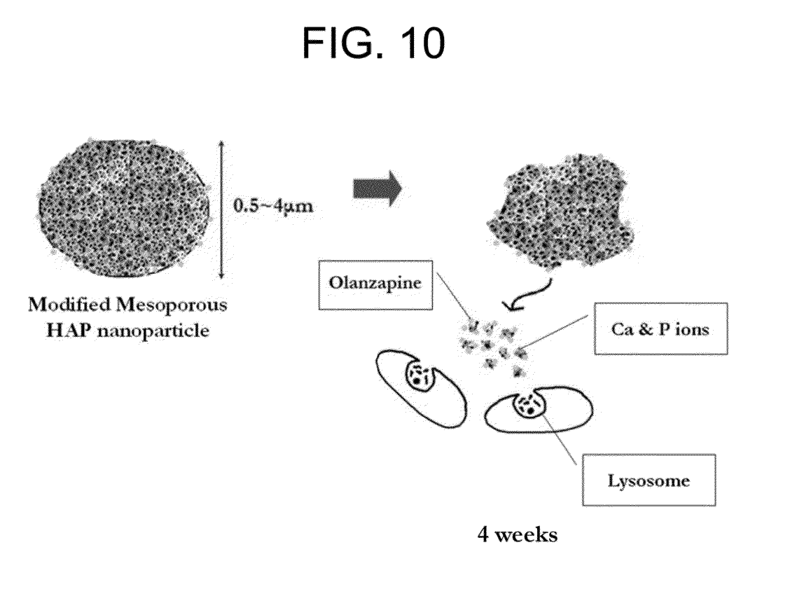

FIG. 10 is a schematic drawing illustrating cellular drug delivery. A composition comprising the antidepressant Olanzapine adsorbed onto mesoporous HAP with hydrophobic surfaces has a particle size of around 0.5-4 μm. After intramuscular injection, the particle enters a cell through phagocytosis. The particles in the cytoplasm are then taken up by lysosomes. The lysosomes degrade the mesoporous HA particles and cause an increase in Ca²⁺ and PO₄ ions, which in turn causes an increase in osmotic pressure within the lysosomes. Lysosomes bust due to influx of H₂O induced by the increased osmotic pressure. Olanzapine is then released out of the lysosomes and enters the cytoplasm. Olanzapine in the cytoplasm are pumped out of the cell. Once outside the cell, Olanzapine may enter the blood stream and slowly circulate throughout the body until reaching its target site. Therefore through the lysosomes, the cell delivers Olanzapine to the blood stream of the patient.

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Materials and Methods

Olanzapine was purchased from Santa Cruz Biotechnology, Inc. (California, USA). Calcium hydroxide, phosphoric acid 85%, and ammonium hydroxide purchased from Riedel-de Haen (Seelze, Germany), Acrylic acid, linoleic acid, potassium persulfate and sodium metabisulfite purchased from Sigma-Aldrich (Wisconsin, USA), and methanol purchased from Merck Chemicals (Darmstadt, Germany).

### Synthesis and Characterization of Mesoporous HAP Nanoparticles

A co-precipitation method was used for synthesizing mesoporous hydroxyapatite nanoparticles (Wu HC et al. (2007) “A novel biomagnetic nanoparticle based on hydroxyapatite. Nanotechnology” 18 (16), 165601, which is incorporated herein by reference in its entirety). Below shows the chemical reaction of the synthesis:

10Ca(OH)₂+6H₃PO₄→Ca₁₀(PO4)6(OH)₂+18 H₂O

0.5 M of calcium hydroxide (Ca(OH)₂) was dispersed as a suspension and kept at 80-85 ° C. in a water bath during the reaction period. A stoichiometric amount (i.e., Ca/P molar ratio=1.67) of 0.3 M orthophosphoric acid (H₃PO₄) solution was added to the Ca(OH)₂ suspension at a rate of approximately 3 ml/min. While the H₃PO₄ solution was titrated into the Ca(OH)₂ suspension, 15 g of albumin was stirred rapidly to form a foam and then was slowly added into the suspension. The pH value of the mixture was adjusted to 8.5˜9 by adding about 2 ml of ammonium hydroxide (NH4OH). The mixture was stirred for 2 hours and then aged for 20 hours while the temperature was kept at 85 ° C. After 20 hours, the mixture was washed once with deionized water and three times with methanol.

The mixture was then placed under vacuum freeze-drying. The dried sample was calcined at 800° C. for decarburization. The crystallinity of the synthesized mesoporous HAP powders (nanoparticles, nanocomposites, or composites) was identified using X-ray diffractometer (Rigaku, USA), and the diffraction angle was set in the range of 10-60° at a scan rate of 1° /min. The surface morphology of the synthesized mesoporous HAP nanoparticles was identified using a scanning electron microscope (Philips, USA).

Afterwards, a series of surface modifications were performed by adding acrylic acid and linoleic acid. The HAP powders with a net weight of 2.5 g were vigorous stirred with 100 ml of deionized water, and then degassed by N₂ bubbling for 30 minutes to obtain a degassed HAP suspension. Redox initiators (i.e., catalysts) of potassium persulfate (K₂S₂O₈, 0.01 g, 0.38×10⁻⁴ mol) and sodium metabisulfate (Na₂S₂O₅, 0.01 g, 0.38×10⁻⁴ mol) were rapidly stirred with 1 ml of deionized water and then added into the aforementioned degassed HAP suspension to obtain a mixture comprising the HAP and the catalysts. Acrylic acid (C₃H₄O₂, 2.16 g, 3×10⁻² mol) was added into the aforementioned mixture. The pH of the reaction mixture was adjusted to 9.5 with ammonium hydroxide (˜9 ml). The reaction was performed at room temperature under constant stirring and N₂ atmosphere for 3 hours. The mixture was then centrifuged at 3000 rpm for 10 minutes and washed with deionized water for 3 times before it was placed under vacuum freeze-drying. The following chemical reactions were considered to have occurred during the surface modification by acrylic acid.

The surface modification of mesoporous HAP was performed by grafting free radicals of acrylic acid in the presence of equimolar K₂S₂O₈/Na₂S₂O₅ redox initiators. The possible mechanism of the reaction is shown below. S₂O₈²⁻ is ultimately decomposed to 2SO⁴⁻ in the presence of S₂O₅²⁻. In addition, OH⁻ and SO⁴″ radicals induce active radicals on HAP nanoparticles by removing hydrogen atoms from the surface of mesoporous HAP nanoparticles. As the amount of grafted acrylic acid increases, the surface areas of mesoporous HAP increases as well.

(1) Initiation: Persulfate Decomposition in the Aqueous Phase

S₂O₈²⁻+S₂O₅²⁻→2SO₄⁻

2SO*₄⁻2H₂O→2*OH+2SO₄+2H⁺

(2) Radical Formation on HAP Nano-Surfaces

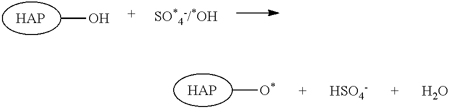

After the above procedures were completed, the acrylic acid-surface modified mesoporous HAP powders with a weight of 125 mg were vacuumed at 1×10⁻⁴ torr for 5 hours. Approximately 1˜2 ml of linoleic acid (C₁₈H_{32O}₂) was then added into the system (i.e., the acrylic acid-surface modified mesoporous HAP powders under vacuumed environment) with a syringe and stirred rapidly for 12 hours. OLZ (50 mg) was dissolved in about 1˜2 ml deionized water and added into the above system (i.e., the acrylic acid-surface modified mesoporous HAP powders added with linoleic acid under vacuumed environment) with rapid stirring for 12 hours (Gardner I et al. (1998) “A Comparison of the Oxidation of Clozapine and Olanzapine to Reactive Metabolites and the Toxicity of these Metabolites to Human Leukocytes” Molecular Pharmacology, 53 (6), 991-998). After the linoleic acid reaction, mesoporous HAP without loading any drug could be stored under vacuum freeze-drying.

### Drug Loading

OLZ was physically adsorbed onto the surface-modified mesoporous HAP. To determine the amount of OLZ entrapped in the surface-modified mesoporous HAP, all the organic materials had been calcined at up to 600° C. under N2 atmosphere to obtain the TGA-DTA results of (1) mesoporous HAP, (2) surface-modified mesoporous HAP, and (3) surface-modified mesoporous HAP incorporated with OLZ, respectively. The heating procedure in the TGA-DTA measurements was as follows:

(1) heating to 100° C. with a rate of 5° C./min;

(2) isothermal for 20 min;

(3) heating to 600° C. with a rate of 5° C./min; and

(4) isothermal for 20 min.

The mixture of OLZ and surface-modified mesoporous HAP nanoparticles (i.e., the OLZ-loaded surface-modified mesoporous HAP) was obtained by centrifuging the nanoparticles suspension at 1000 rpm for 10 minutes, and the final product was around 155 mg with the assumption that there was no loss in HAP during centrifugation. The ratio of LA to OLZ is 6.5:3.5 as determined by the loss in weight during the TGA-DTA analysis. The drug entrapment was calculated by using the following formula (Vandervoort J and Ludwig A. (2004) “Preparation and evaluation of drug-loaded gelatinnanoparticles for topical ophthalmic use” European Journal of Pharmaceutics and Biopharmaceutics, 57, 251-261).

$$Drug entrapment ( \% ) = \frac{mass of drug in nanoparticles \times 100}{mass of drug used in formulation}$$

The mass of drug used in formulation was 50 mg used for drug-loading.

### In Vitro Cytotoxicities

Mouse embryonic fibroblast 3T3 cells were used for evaluations of cytotoxicities of HAP-AA-LA-OLZ nanoparticles. The cells were cultured in 10 ml of Dulbecco's modified eagle's medium (DMEM) supplemented with 10% fetal bovine serum (FBS). The cells were incubated with various concentrations of HAP-AA-LA-OLZ nanoparticles (0, 0.05, 0.1, and 0.5 mg /ml) for 24 and 72 hours in an incubator at 37° C. and 5% CO₂ air. Each concentration had six replicates (N=6). The cell survival was evaluated using LDH and WST-1 assays and the light absorption was determined at 420 nm on an ELISA reader. The relative cell survival in the presence or absence of the HAP-AA-LA-OLZ nanoparticles was calculated.

### In Vitro Drug Release Study

Olanzapine-loaded surface-modified mesoporous HAP (HAP-AA-LA-OLZ) was placed in separate containers with 10 ml phosphate buffered saline (PBS) solution at pH 7.4 at 37° C. in the incubator. The solutions were collected at pre-determined time points. After being passed through a 0.22 μm filter, the absorption of the collected solution at 226 nm was measured with a UV spectrophotometer (JASCO V-670). The containers were re-filled with 10 ml of fresh PBS after each collection. The absorption in the sample was compared with a standard curve for determination of the released drug concentration in the solution. A graph of in vitro drug release profile was plotted.

### In Vivo Drug Release Study

Male ICR mice (31-33 g) were purchased from BioLasco Taiwan Co., Ltd. All animal experiments were conducted in accordance with the Guide for the Care and Use of Laboratory Animals published by the National Institute of Health. The mice were given a defined amount of drug via intramuscular injections and sacrificed at intervals over 5 weeks and blood samples collected. Serum was obtained by centrifugation of the blood samples at 800 rpm for 10 minutes and the absorption at 252 nm measured. The absorption in the serum sample was compared with a standard curve for determination of the released drug concentration. A graph of in vivo drug release profile was plotted.

### Results

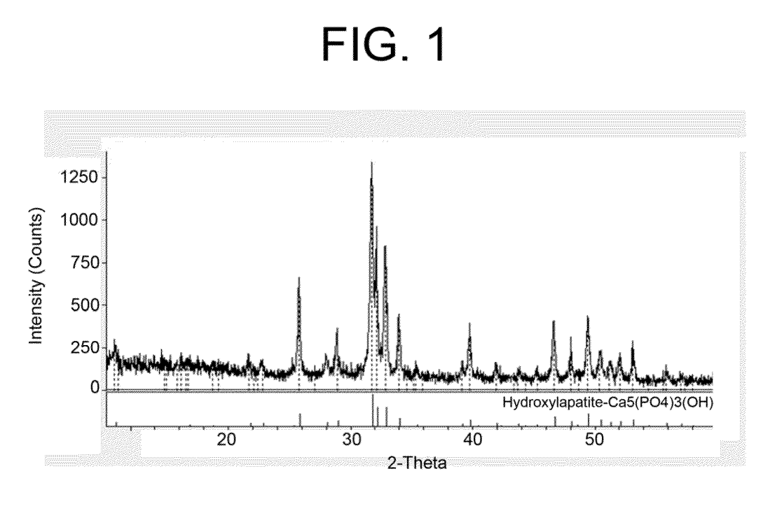

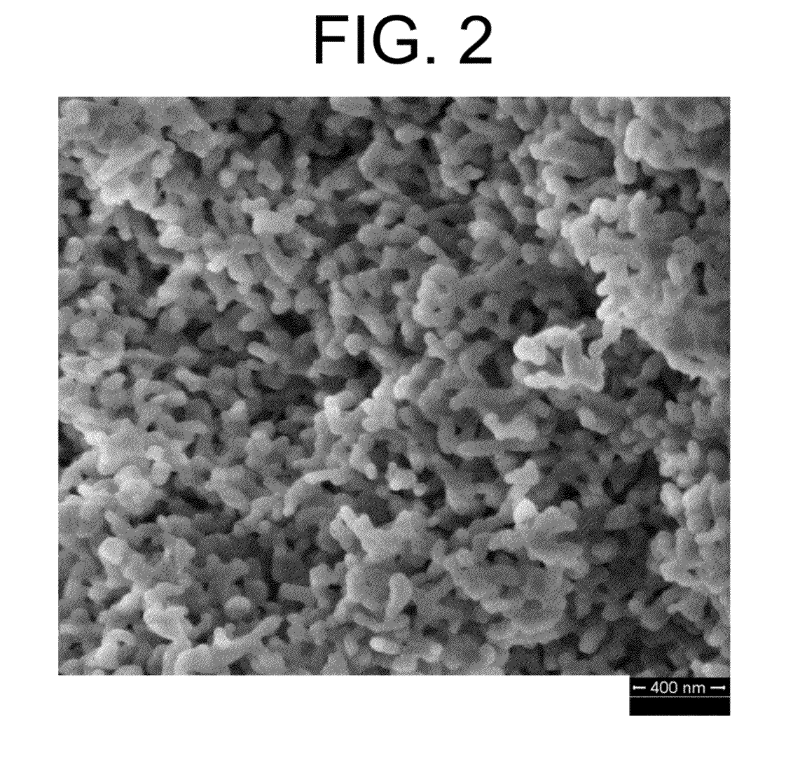

Mesoporous HAP particles without surface modifications by acrylic acid and linoleic acid were examined using X-ray diffractometer (Rigaku, USA) to determine the properties. FIG. 1 shows three main peaks at 31.68°, 32.12°, and 32.82° in the XRD pattern. For comparison, the XRD pattern of HAP purchased from JCPDS (Catalog number 09-0432) is shown at the bottom of FIG. 1, indicating that our mesoporous HAP had an XRD pattern corresponding to the standard pattern. Thus, it was concluded that the mesoporous HAP particles synthesized were indeed HAP. The surface morphology of mesoporous HAP particles was examined with SEM. The image (100 000×) in FIG. 2 shows worm-like mesoporous structure within the particle. The size of mesoporous HAP (surface modified) was measured by a particle size analyzer. The particle size ranged from 435 to 5174 nm (data not shown) with a majority of the particles falling in the range of 400˜500 nm.

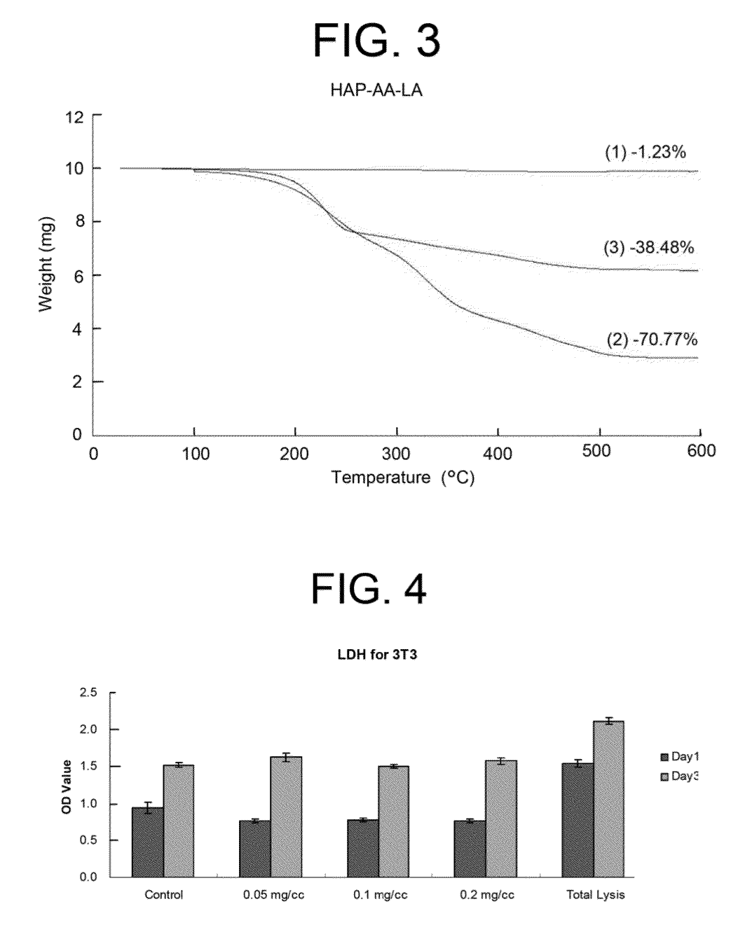

FIG. 3 shows the TGA-DTA results of (1) mesoporous HAP with a weight loss of 1.23%, (2) surface-modified mesoporous HAP with a weight loss of 70.77%, and (3) surface-modified mesoporous HAP incorporated with OLZ with a weight loss of 38.48%. The average mass of drug in the nanoparticles was 10.395 mg (N=6), calculated as follows: (155-125.2)×0.3488=10.395.

The mass of drug used for preparing the formulation was 50 mg. Therefore, the drug entrapment calculated was 20.79%. In other words, 50 mg of OLZ was added, but only 10.395 mg was attached or loaded onto the mesoporous HAP.

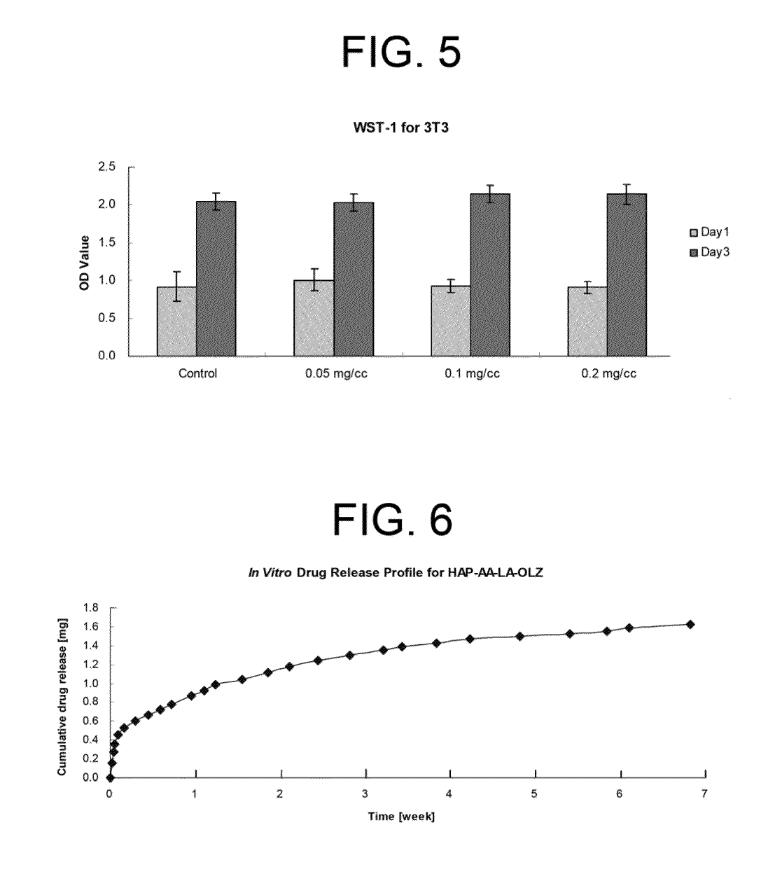

FIG. 4 shows the results of LDH assay of 3T3 cells for cytotoxicity on Days 1 and 3. There was no significant difference in the OD values between the control (0 mg/ml) and experimental groups (0.05 mg/ml, 0.1 mg/ml, and 0.2 mg/ml). It was concluded that HAP-AA-LA-OLZ particles had a relative low cytotoxicity to the cells. FIG. 5 shows the results of WST-1 assay of 3T3 cells for viability and cell proliferation, indicating that the HAP-AA-LA-OLZ nanoparticles had no negative effects on viability and cell proliferation on the cells after Days 1 and 3.

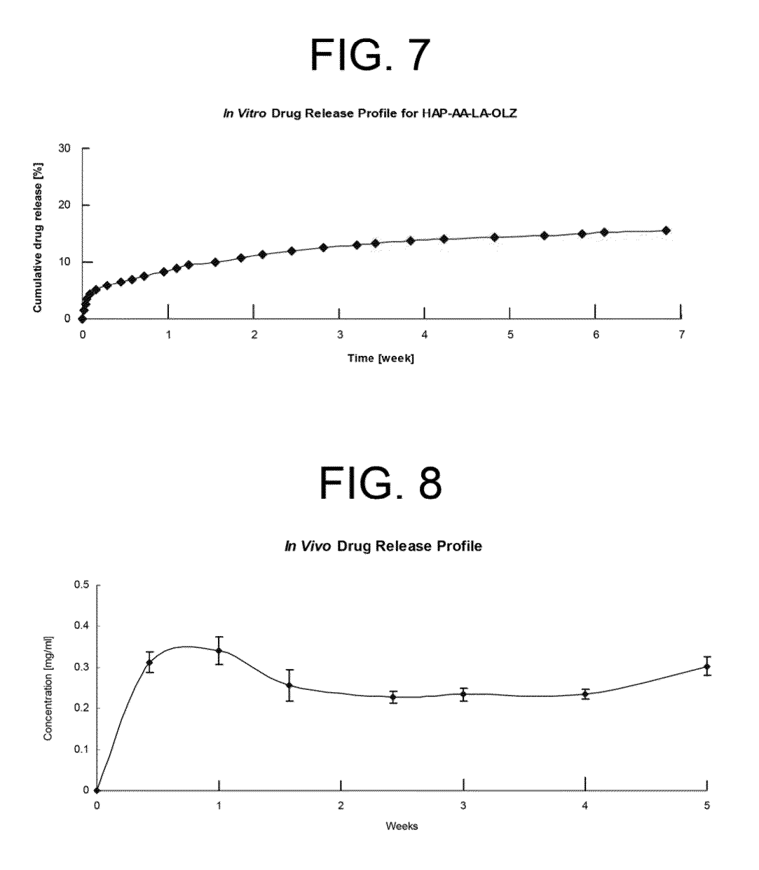

FIGS. 6 and 7 show in vitro cumulative mass and percentage release profiles of OLZ from mesoporous HAP nanoparticles over a 7-week period, respectively. The data showed a low initial burst release (5.12% of 10.395 mg drug) after 24 hours, an ideal, long-lasting intramascular dosage form for an antidepressant. The cumulative drug releases on weeks 4 and 7 were 1.467 mg (14.11%) and 1.625 mg (16.63%), respectively. The data indicated surface-modified mesoporous HAP nanoparticles according to the invention prolonged OLZ release in vitro for up to 7 weeks.

FIG. 8 shows an in vivo drug release profile of OLZ from mesoporous HAP nanoparticles over a 5-week period. Initial burst occurred in the first week after IM injection, which was consistent with the report that OLZ reaches a steady-state in one week after the first dosage. On week 5, it was noticed that the drug concentration was trending upward, which might result from uptake and breakdown of HAP-AA-LA-OLZ by lysosomes in cells such as macrophages.

In summary, mesoporous HAP nanoparticles were synthesized by using a co-precipitation method, and the mesoporous structure was identified using SEM images. The particle size was in the range of 500˜3700 nm, which was adequate for cellular uptake. A series of surface modifications with acrylic acid and linoleic acid were made on the mesoporous HAP and then OLZ was incorporated. LDH and WST-1 cytotoxicity assays indicated that the HAP-AA-LA-OLZ has low cytotoxicity to cells. The in vitro drug release profiles showed a low initial burst (5.12%) and a steady constant release of drug over a 7-week period. The in vivo drug release profile showed an initial burst occurred at the first week and a steady constant release was also observed for at least 5 weeks.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

1. (canceled)

2. (canceled)

3. A method of producing a cellular drug release, comprising:
(a) providing a composition comprising a therapeutically effective amount of a pharmacological agent loaded onto mesoporous hydroxyapatite (HAP) with hydrophobic surfaces;
(b) exposing a cell to the composition;
(c) causing entry of the mesoporous HAP into the cell and dissolving of the mesoporous HAP in the lysosomes of the cell and release of the pharmacological agent from the mesoporous HAP;
(d) causing the pharmacological agent to release from the lysosomes and enter the cytoplasm; and
(e) causing the pharmacological agent to be pumped out of the cell.

4. The method of claim 3, wherein the pharmacological agent comprises an antidepressant.

5. The method of claim 4, wherein in step (e) the pharmacological agent is released into the blood stream of the animal.

6. The method of claim 5, wherein the pharmacological agent is released continuously into the blood stream of the animal continuously for a period of 4 weeks or longer than 4 weeks without an intermittent cessation.

7. The method of claim 6, wherein the pharmacological agent is released into the blood stream of the animal continuously for a period of 5 weeks or longer than 5 weeks.

8. The method of claim 3, wherein the cell is present in an animal and exposed to the composition via intramuscular injection of the pharmacological agent to the animal.

9. The method of claim 4, wherein the cell comprises neutrophils, monocytes, macrophages, dendritic cells, and mast cells.

10. The method of claim 3, further comprising causing an increase in Ca²⁺ and PO₄³⁻ ions within the lysosomes of the cell.

11. The method of claim 3, wherein the mesoporous HAP comprises HAP particles with each particle having a size of ranging from 500 to 3700 nm.

12. The method of claim 3, wherein the mesoporous HAP with hydrophobic surfaces comprises:
a) mesoporous hydroxyapatite (HAP);
b) acrylic acid, grafted onto the surface of the mesoporous HAP and forming an acrylic acid-grafted mesoporous HAP; and
c) a pharmaceutically acceptable amphiphilic compound, modifying the surface of the acrylic acid-grafted mesoporous HAP and forming hydrophobic hydrocarbon tails on the surfaces thereof.

13. The method of claim 12, wherein the amphiphilic compound comprises a fatty acid from between 10 to 40 carbon atoms.

14. The method of claim 13, wherein the fatty acid is selected from the group consisting of capric acid, lauric acid, myristic acid, palmitic acid, palmitoleic acid, stearic acid, isostearic acid, elaidic acid, oleic acid, linoleic acid, polyunsaturated elaidolinoleic acid, polyunsaturated linolenic acid, elaidolinolenic acid, polyunsaturated ricinoleic acid, arachidic acid, behenic acid, erucic acid, lignoceric acid, ceric acid, montanic acid, melissic acid, and geddic acid.

15. The method of claim 12, wherein the amphiphilic compound comprises linoleic acid.

16. The method of claim 3, wherein the mesoporous hydroxyapatite (HAP) contains no water-soluble polyvalent metal compound, and/or calcium as a constituent of hydroxyapatite is not substituted by other metal.

17. The method of claim 12, wherein the amphiphilic compound comprises a pharmaceutically acceptable surfactant.

18. The method of claim 17, wherein the pharmaceutically acceptable surfactant comprises a polyoxyethylene glycolated natural or hydrogenated vegetable oil, or hydrogenated castor oil.

19. The method of chum 17, wherein the pharmaceutically acceptable surfactant is at least one selected from the group consisting of sodium lauryl sulfate, polyoxyethylenesorbitan monolaurate, cetyltrimethylammoniumbromide, polyoxyl castor oil, hexadecyltrimethylammonium bromide, polyethylene glycol tert-octylphenyl ether, nonylphenol ethoxylate, cyclodextrins, and lecithin.

20. (canceled)

21. The method of claim 3, wherein the composition comprises:
(a) mesoporous hydroxyapatite (HAP);
(b) acrylic acid, grafted onto the surfaces of the mesoporous HAP and forming an acrylic acid-grafted mesoporous HAP;
(c) linoleic acid, modifying the surfaces of the acrylic acid-grafted mesoporous HAP and forming hydrophobic hydrocarbon tails on the surfaces thereof; and
(d) a therapeutically effective amount of an antidepressant loaded onto the hydrophobic hydrocarbon tails.

22. The method of claim 21, wherein the composition exhibits the following characteristics:
(i) release of less than 10% of the antidepresant from the mesoporous HAP in a fluid having a pH value of about 7.4; and
(ii) release of the antidepresant in a fluid having a pH value of about ≦5.

23. The method of claim 21, wherein the composition comprises a therapeutically effective amount of a pharmacological agent loaded onto mesoporous hydroxyapatite (HAP) with hydrophobic surfaces.
